(19) United States
(12) Patent Application Publication
(10) Pub. No.: US 2010/0209360 A1
(43) Pub. Date: Aug. 19, 2010

(54) Method for Making a Gas from an Aqueous Fluid, Product of the Method, and Apparatus Therefor

(75) Inventors: Ted Suratt, Clearwater, FL (US); Robinson Burroughs Gourley, Sarasota, FL (US)

Correspondence Address: Eugenia S. Hansen;Hemingway & Hansen, LLP, Comerica Bank Tower Suite # 2500, 1717 Main Street, Dallas, TX, 75201, US

(73) Assignee: LSG HOLDINGS, INC., Sarasota, FL (US)

(21) Appl. No.: 12/596,077

(22) PCT Filed: Apr. 17, 2008
(86) PCT No.: PCT/US08/60666

§ 371 Date: Mar. 17, 2010

Related U.S. Application Data
(63) Continuation-in-part of application No. 11/738,476, filed on Apr. 21, 2007.

(51) Int. Cl. A61Q 11/00; C25B 5/00; C01B 13/00; A61K 33/00; C12N 5/00; F02M 25/00

(52) U.S. Cl. 424/49; 204/156; 423/579; 424/600; 435/375; 435/410; 123/585

(57) ABSTRACT

A method for producing a purified, stable, compressible gas from an aqueous fluid. The gas is suitable for a variety of uses and may also be infused into water which itself is useful for a variety of purposes.

### CROSS REFERENCE TO RELATED APPLICATIONS

This is a continuation-in-part and claims the benefit of, copending U.S. patent application Ser. No. 11,738,476 filed on Apr. 21, 2007.

### STATEMENT REGARDING FEDERALLY SPONSORED RESEARCH OR DEVELOPMENT

Not applicable.

### TECHNICAL FIELD

This invention relates to the generation of a purified stable gas from an aqueous fluid, wherein said gas may be stored under pressure and uses for the gas.

### BACKGROUND OF THE INVENTION

Electrolysis of water is known to produce hydrogen gas (H₂) at the cathode and oxygen gas (O₂) at the anode. Due to the high heat of the chambers, water vapor also resulted from this process. If the hydrogen gas and oxygen gas were not effectively separated, such methods resulted in an impure gaseous product that could not be effectively compressed or stored under pressure for industrial applications in a single container, and was deemed explosive and dangerous. Thus, it remained desirable to develop a method by which a useful, stable, purified, compressible single gas could be formed from water or an aqueous fluid.

### BRIEF DESCRIPTION OF THE DRAWINGS

FIG. 1 illustrates a schematic of a preferred reaction chamber for the invention.

FIGS. 2-3 illustrate the inventor's conception of the nature of the gas as formed from the process disclosed herein.

FIG. 4 illustrates graphs showing the absorption of Vitamin C by cells treated with SG Gas-infused Water and control. FIG. 4A shows the effect on basolateral cells and FIG. 4B on apical cells.

FIG. 5 illustrates properties of SG Gas-infused Water.

### DETAILED DESCRIPTION

A method for generating a gas having desirable properties is herein disclosed. In addition, methodology for purifying said gas is disclosed. Applicants refer to this gas as “SG Gas.”

Applicant hereby incorporates U.S. Ser. No. 11/738,476 filed on Apr. 21, 2007 by reference as if fully set forth herein.

In a first step of the method, an aqueous fluid is provided to a reaction zone. While various aqueous fluids, such as distilled water, tap water, or water taken from a river, stream, lake or the like may be used to generate electrical current at satisfactory levels, it is preferred to use an electrolyte solution for the aqueous fluid of standardized composition so that the conditions of the method can be better standardized for maximum yield of gas.

The aqueous fluid is provided to a reaction zone which is preferably closed off so to allow the reaction to occur under pressure. An alkali salt is preferably used as an electrolyte dissolved in distilled water. Preferred alkali salts are potassium hydroxide, lithium hydroxide and sodium hydroxide. The specific gravity of the alkali salt in the solution is above 1.0. Most preferably, potassium hydroxide is employed at a specific gravity from at least above 1.0 up to about 1.2. If another electrolyte is chosen other than potassium hydroxide a mole ratio must be calculated for that substance so that the maximum mole ratio represented by the specific gravity of 1.2 provided for potassium hydroxide will not be exceeded. These specific gravity values are as determined by a refractometer which provides readings that are temperature compensated. Most preferably, the electrolyte employed is potassium hydroxide (powder form) dissolved in distilled water at a concentration sufficient to form a solution having up to 1.2 specific gravity. A suitable refractometer is the Westover Model RHA-100, portable refractometer.

Aqueous fluid is contained in a receptacle which can be made out of a variety of materials including sheet steel, stainless steel, CV-PVC and epoxy resin fiberglass. The apparatus and internal devices need to be heat resistant and waterproof. The reaction zone is comprised of said aqueous fluid.

The aqueous fluid is placed in a reaction zone in the method of the invention. Overall, the method employs creation of a magnetic field in the aqueous fluid and periodic collapse of the magnetic field under conditions which do not provoke electrolysis of the aqueous fluid. Under these conditions, a single gas is generated and collected. This gas has desirable properties and is useful for applications.

In a first step of the method, a magnetic field is applied to the reaction zone. Preferably, the magnetic field is applied by providing a source of electric power to said reaction zone. An electric current in said reaction zone provides a magnetic field.

In a preferred embodiment, two metallic end plates having an inside surface and an outside surface, and having the capacity to conduct an electrical current are used in the reaction zone in opposing configuration. The inside of each end plate is partially submerged in the electrolyte solution. The metallic plates are preferably comprised of nickel alloy or stainless steel, but any metal can be used as long as such metal has the capacity to conduct an electric current and is preferably resistant to erosion by alkali solutions. One of said metallic plates serves as a cathode and the other as an anode. The cathode and anode should be separated a sufficient distance so that a magnetic field forms when current is applied to the reaction zone. The distance between the plates must be greater than one inch (2.5 cm) in the method of the invention and is preferably eight to sixteen inches apart. This distance is independent of the volume of the aqueous fluid employed or size of the reaction zone.

There is a relationship between the concentration of electrolyte solution and the amperage which will exist in the aqueous fluid upon application of current thereto. The higher the specific gravity, the greater the amperage will result. This will also affect the strength of the magnetic field, and increase the temperature of the solution. Electrolysis (used industrially to produce hydrogen gas via the reaction 2H₂O(l)→2H₂(g)+O₂(g)) which is not desired in the method of the invention, could occur if the current is too high. The current may be too high if the specific gravity of the electrolyte exceeds the equivalent of 1.2 for potassium hydroxide.

In order for the magnetic field to be applied to the reaction zone, a power source (e.g., 110 volts DC) is applied respectively to the anode and to the cathode.

An appropriate power source that may be used in the method of the invention is 110 volt alternating current which has been converted to direct current using a rectifying process (e.g., a diode bridge device). Any standard power or voltage source may be used as long as it is rectified to direct current. When an electric current is applied to the reaction zone, a magnetic field is created in the reaction zone, which periodically collapses and causes the conversion of the water in the aqueous fluid into gas. Cyclic pulsation will be present in current even after alternating current is converted to direct current (for example a 60 cycle pulsation from household current) unless a smoothing circuit has been incorporated. This resulting cyclic pulsation is employable in the invention to periodically collapse the magnetic field, however using an auxiliary pulsing unit is preferably used in the method of the invention so that better regulation of pulsing may be employed. Any means for causing the electric current provided to the reaction zone to pulse at a frequency of 15 to 20 kilohertz decreases the wattage needed to create gas by approximately a factor of 10. The amount of energy needed to generate one (1) liter of gas is 0.0028 kilowatt-hour and with a pulsing device associated with the reaction zone, the amount drops to 0.00028 kilowatt-hour or less to generate one (1) liter of gas.

As the pulsing occurs, the stationary magnetic field alternatively collapses and is reinstated. It has been found that a reaction occurs in the electrolyte solution between the two end plates upon collapse of the magnetic field, which results in a release of a generated gas. Some of the same gas will be pulled toward the individual plates and released as part of the generated gas.

In a pilot plant apparatus for determining optimal conditions, a clear Plexiglas receptacle can be used for the reaction zone, so that one can visibly monitor the reaction with ultraviolet light and observe the generation of gas. This pilot plant preferably provides adjustment means for the cathode and anode so that they can be moved to optimize the reaction for a given aqueous fluid composition and changes in pulsing duration and frequency.

Gas is generated not only at the electrodes but also appears as bubbles in the body of water between the electrodes. It has been found that use of minimal electric currents between two electrodes results from the electrodes being spread a sufficient distance apart of at least one inch (2.5 cm) and preferably eight to sixteen inches apart, thereby creating the aforesaid magnetic field enveloping the reaction chamber. A pure gas is produced in the body of aqueous fluid between the electrodes, without the production of a high levels of heat that would cause the water to vaporize (212° F.). Rather, the reaction zone remains at a temperature not exceeding 120° F. dependent on ambient temperature. Normally, there is a 30° F. temperature rise above ambient temperature assuming room temperature 90° F. The collection chambers contain no increase in oxygen gas, no increase in hydrogen gas, and no noticeable water vapor. Thus, costs are lowered, production speed increases, and the resulting gas is uniform in its properties. Also important, the resulting homogeneous gas can be pumped into a stainless steel cylinder and has been found to be stable and not explosive under pressures of over 1000 lb.

The important functionalities in the process are imposition of a magnetic field on the aqueous fluid and the ability to periodically collapse the magnetic field to generate the desired gas, under conditions short of those that will induce electrolysis. Other means which provide for these functionalities can be used. For example, in an alternative embodiment, wires could be inserted instead of plates in the reaction zone and when current passes from one wire through the aqueous fluid to the other wire, a magnetic field would be produced. In another exemplary alternative, a wire coil outside the reaction zone could be used to which a source of DC power can be supplied to create a primary magnetic field in the reaction zone. A wire coil placed in the middle of the solution can serve as a secondary magnetic field and when powered in the opposite direction of the current flow in pulses would collapse the primary field and create the necessary reaction to form the gas. Such a coil would be similar in concept to an automobile coil.

When water is converted into gas, the natural conversion from liquid to gas creates an increase in volume and thus an increase in pressure within the reaction zone. While standard atmospheric pressure is about 14.7 psi at sea level, the pressure in the closed reaction zone is maintained between 30 and 100 psi by using a check valve at the outlet of the reaction chamber to control it, since maximum gas production occurs in this pressure range.

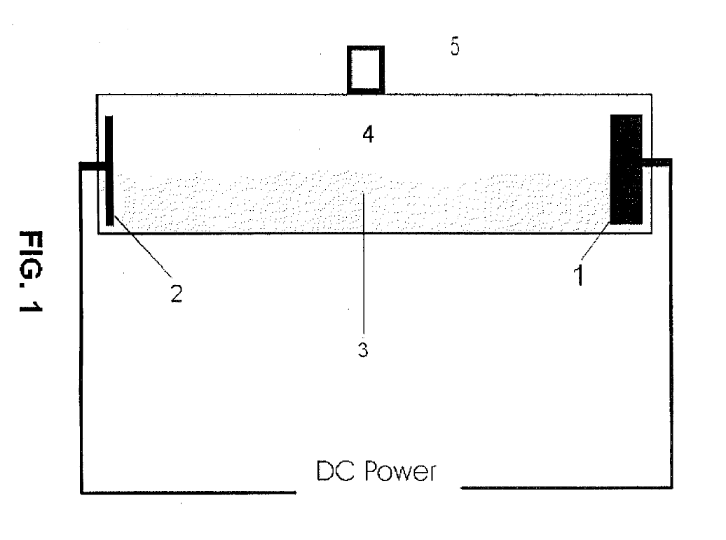

Now referring to FIG. 1, a schematic of a reaction chamber is illustrated. Cathode (1) and anode (2) are in opposing configuration, preferably more than one inch apart and most preferably eight to sixteen inches apart. In the process of the invention, a current is passed through an aqueous fluid (3) and the current flow through the electrolyte creates a magnetic field. The electricity is pulsed, which collapses the magnetic field with each pulse of electricity. This produces the gas at a very efficient rate in the area of the solution between the electrodes, as denoted by (4) in FIG. 1. The gas produced may be collected from the reaction zone through gas outlet (5) and subjected to further purification as taught herein.

The generated gas is then preferably exposed to a second magnetic field by providing a second reaction zone comprising of rare earth magnets. The strength of the rare earth magnets should be greater than fifty (50) Gauss units. Gas flows through a chamber exposed to rare earth magnets for purification. Rare earth magnets, dense metal magnets typically made from a composite of neodymium, iron and boron with or without a nickel coating or plating, are attached to the exterior of the chamber. Since SG Gas is paramagnetic and water vapor is diamagnetic the magnetic chamber strengthens the molecular bond of the gas and repels the water vapor back into the solution.

The purified SG Gas may be used immediately or compressed and stored in a gas storage tank. Purified SG Gas may be allowed to flow out of said second reaction zone directly to a torch attachment, to a compressor for storage in a pressurized vessel, or gas outflow valve for infusion into water or other substances.

In a method for making a compressible, stable gas with desirable properties, SG Gas is made according to the method of the invention. SG Gas can then be safely compressed and stored. SG Gas can be compressed above 1,000 psi. SG Gas also can be stored in a pressurized vessel.

In an exemplary procedure for compression, SG Gas is discharged from the apparatus into a hose with a compressor attached. We use a Whirlwind Compressor, Model 2200-2 HPE, manufactured by High Pressure Eng. Co., Inc. A canister with pressure gauges is used to fill the chamber with SG Gas, using a hose to transport the SG Gas from the apparatus and compressor into the canister. We use an empty oxygen tank that has been vacuumed to remove any residual oxygen and water. The empty and vacuumed oxygen tank with pressure valve has a manufacturer name of White Martins, ABRE with dimensions of 23″ diameter and 19″ height. SG Gas is placed under pressure in the compression chamber up to and beyond 1,000 psi. for storage of SG Gas.

SG Gas remains stable and under pressure for one month and longer. To test its stability, wood chips were placed in a stainless steel tank and the tank filled with SG Gas. The wood chips absorbed SG Gas and additional SG Gas was used to refill the chamber and maintain a 30 psi. Once the wood chips were saturated with SG Gas, the tank was decompressed and pressure reduced to 0 psi. For a period of over 30 days, no pressure was generated assuming that no out gassing of SG Gas occurred. The wood chips displayed different burn properties after 60 days when compared to that of the non-treated wood chips. The treated wood chips with absorbed SG Gas burned more efficiently when compared to that of non-treated wood chips thereby demonstrating the stability of the SG Gas bond with the treated wood chips.

Analytical Testing and Observations of SG Gas Under Pressure Maximum Pressure: SG Gas imploded when pressures exceeded 1,600 psi. Safe Pressurization: SG Gas remains safe and stable at pressures around 1,000 psi for over 30 days. SG Gas should remain stable under pressure indefinitely, at least for a sufficient period of time to allow said gas to be utilized at time 30-60 days after generation.

The purified SG Gas was tested and exhibited properties of a pure, homogeneous gas that was found to be compressible as stated above, safe, also able to oxidize any non-oxidized substrate its flame contacts and able to reduce any completely oxidized substrate its flame contacts. The following characteristics were observed.

Ultra-violet Light Test: Exhibits a blue gray color appearance compared to untreated distilled water which exhibits no color, when exposed to an ultra-violet light, manufactured by Zelco Industries Model 10015.

Balloon: Is lighter than air and causes balloons filled therewith to rise.

Cooling: The Balloon Filled with Purified Gas: Balloon remains inflated at or below −10° F.

Ignition: The purified SG Gas produced according to the above method was tested for ignition properties. The purified gas, when lit with an ignition source such as a spark, causes an implosion. The temperature of the flame produced upon ignition was estimated to be about 270° F. using an infrared temperature device (Raynger ST2L infrared temperature device). However, when materials are exposed to the flame, which creates a chemical reaction with the material, base metals will rapidly rise to melt temperature points, releasing heat and converting the gas back into water (H₂O).

Purified SG Gas was discharged from the reaction zone through a hose with a torch attached. On the gas output of the apparatus, a flash-back arrestor is recommended. The gas may be exposed to an ignition source (e.g., spark or electrical arc) thus combustion of the gas occurs. The heat of the resulting flame on the subject torch has a temperature of approximately 270° F.

When an air/propane torch is burning, a small amount of SG Gas is introduced into the air mixing chamber of a lit propane torch, a single uniform flame cone becomes visible demonstrating a more efficient conversion of hydrocarbon and more heat from combustion of hydrocarbon, meaning it has a use as a fuel extender. One use is injection of SG Gas into an air intake of a combustion engine thereby reducing harmful exhaust emissions and increasing fuel efficiency. A by-product of this process is the creation of water during the combustion cycle that generates steam. The steam causes an increase in the torque generated by the engine resulting in greater power output. Depending upon the type of fuel, SG Gas extends fuel efficiency by a factor between 2 and 10.

When ignited purified gas contacts another substance, melting occurs within a short period of time, usually less than one minute. The results of some examples of substances exposed to ignited purified SG Gas may be found in Table 1.

TABLE 1
Effect of Ignited Purified Gas on Various Substances

| | | | Effect on Exposure to |
|---|---|---|---|
| | | | Ignited Purified SG |
| | | | Gas (one minute or |
| Substance | Melting Point | | less). |
| Stainless Steel | 2,600° | F. | Melting. |
| Steel | 1,330° | F. | Melting. |
| Copper | 1,984° | F. | Melting. |
| Ceramic | 10,000° and 12,000° | F. | Melting. |
| Tar Sands | | | Sand converted to |
| | | | glass and metals were |
| | | | separated out of the |
| | | | sand matrix. |
| Concrete | | | Creates a glassy |
| | | | molten surface which |
| | | | can adhere to metal |
| | | | when cooled. |
| Glass | | | Melts. Flame and |
| | | | true colors are |
| | | | achieved with no |
| | | | carbon flakes or |
| | | | residue embedded |
| | | | inside the glass. |

In lieu of melting a substrate, ignited purified gas may be applied to a substrate with a view toward capturing the generated heat as a useful product. The heat generated can be transferred to a substance such as air or water, thereby producing hot air or steam that can then be used industrially, such as for example to drive a turbine or piston-type engine for production of mechanical energy. In a preferred method, the flame of the SG gas can be applied to a substrate in conduit form having an inside surface and an outside surface. A substance such as forced air or water can flow thorough the conduit adjacent the inside surface of the conduit. The flame of the SG gas can be applied to the outside surface of the conduit which causes the heat-generating reaction to occur. The heat is then transferred to the substance flowing through the conduit, preventing melting of the surface but creating a useful heated fluid that can be used in further applications. An exemplary conduit is a metal tube or pipe, such as copper tubing. It has been further determined that SG Gas can be infused into other substances, rendering a useful product.

Candles: SG Gas infused into melted paraffin wax and poured into a mold with a wick will create candles that burn with lower carbon emission as observed using a Pace 400 Four Gas Analyzer.

Fluids: The gas had an affinity for water and other liquids including fuels but bubbled from the liquids after reaching a saturation point. One novel use of the gas is infusing it back into water to create ionized or polarized water. The resulting gas-infused water creates smaller water clusters that are believed to permit faster cellular absorption and hydration.

In an exemplary method for infusing SG Gas into water, SG Gas is discharged from the reaction zone into a hose with a ceramic diffuser attached. For treating large volumes of water, a ceramic block diffuser may be used. The diffusers are used to reduce the size of the SG Gas bubbles to improve efficiency of water absorption. SG Gas may also be stored under pressure, then infused into water.

It is preferred to infuse water that has gone through a distillation process prior to infusion of SG Gas into treated water with less than 1 ppm TDS (Total Dissolved Solids). One may use an absorption graph to determine time required for achieving desired absorption of SG Gas into water. The typical rate of 30% absorption is approximately one hour to treat 100 gallons of water. A higher saturation of SG Gas up to 100% of total absorption occurs with more infusion of SG Gas into water over time. The actual time and percentage of absorption of SG Gas are affected by the purity of water, volume of water, size of gas bubbles, temperature and other factors.

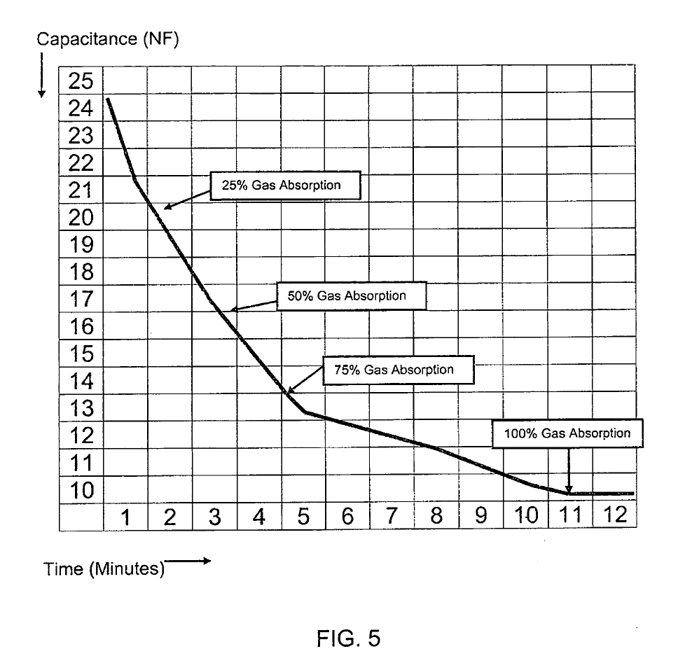

The resulting ionized or polarized water (“SG Gas-infused Water”) clings longer to a magnet when compared to that of regular water. Absorption over time or saturation graphs to monitor changes in the water properties infused with SG Gas including capacitance levels may be prepared. FIG. 5 shows a typical absorption over time graph for infusion of SG Gas into water. Subsequently, one may measure capacitance levels in the treated water over a time period exceeding 30 days to demonstrate that the gas in water is stable. Other measurement: Total Dissolved Solids (TDS) dropped from a start of 0.33 ppm in untreated distilled water to a finish of 0.17 ppm after infusion of SG Gas into distilled water for a period of approximately 11 minutes. A Fluke 189 True RMS Multimeter was used to measure drop in capacitance.

Storage of SG Gas in Water: The resulting polarized water with SG Gas treatment remains stable and can be stored for 2 years or more. The actual maximum storage time has yet to be observed but in theory, SG Gas should remain permanently stable in the water.

Absorption: During infusion of SG Gas into purified water, we used a Fluke 189 True RMS Multimeter to measure drop in capacitance. The absorption over time graph is plotted to monitor the drop in capacitance. The first capacitance drop during initial infusion of SG Gas into a gallon of purified water occurs within the first three minutes of infusion. After that time, the capacitance gradually drops until the point of maximum saturation of SG Gas is typically reached between eight and 20 minutes depending on variables including initial purity of water, size of gas bubbles, and volume of water to be treated. The resulting treated or infused water is referred herein as “SG Gas-infused Water” herein.

Other Parameters Monitored: During infusion of SG Gas into purified water, a drop in TDS (Total Dissolved Solids) concentration, conductivity and resistively can be measured. An appropriate measuring device is a Control Company Traceable™ #4063CC meter.

pH Test: Lab tests show that distilled water had a pH of 6.8 and when infused with SG Gas had a pH change to 7.6.

Ice Cubes: SG Gas remains in SG Gas-infused Water or polarized water until freezing temperatures when the SG Gas forms a gas bubble within the ice cube itself, sometimes producing on the surface of the ice cubes, capillary tubes where the SG Gas escapes.

Ultraviolet Light Exposure: SG Gas-infused Water was tested for the effects of ultraviolet light exposure. A clear spray bottle containing SG Gas-infused Water or polarized water placed in the Florida sun for over two years remained clear in appearance and without algae growth which had been observed in water not infused with SG Gas under similar conditions.

Magnets: A drop of SG Gas-infused Water clings to the surface of a magnet longer when compared to that of untreated water.

Many uses have been found for SG Gas-infused Water. Table 2 lists some of these uses.

TABLE 2
USES FOR SG GAS INFUSED WATER

| | Advantages Provided Over |
|---|---|
| Use | Untreated Water |
| Drinking water for human and | Efficient cellular absorption and |
| animal consumption and hydration | removal of toxins. |
| Water for food and health | Pure form of water that improves |
| supplement manufacturing, | product quality, shelf life, nutrient |
| preparation, and cooking | benefits, absorption, and taste. |
| Water for cleaning and enhancing | Reduced need for emulsifiers and |
| effectiveness of cleansers | surfactants. |
| Water for plants and crops | Greater size of plants, improved plant |
| including hydroponics, floral | quality, longer viability, and reduced |
| arrangements and turf | scale buildup including in hydroponic |
| (golf courses) | water containers. |
| Fertilizer solution for application | Higher yield and more vigorous |
| on plants and crops | growth. |
| Water for aquariums and fish | Greater size of fish. |
| farming | |
| Water systems including long-term | Less algae growth resulting from |
| water storage, municipal supplies | antibacterial properties. |
| and in-home treatment systems | |
| Steam, air heating and air | Less algae or mold growth for cleaner |
| conditioning systems | air circulation systems. |
| Refrigeration systems | Less mold accumulation. |
| Industrial scrubbers | Less algae growth and scale buildup |
| | to maintain scrubbing efficiency. |
| Industrial products and processes | Reduce or eliminate need to use |
| including oil, gas and tar sand | petroleum-based solvents. |
| extraction | |
| Pharmaceutical and medicine | Efficient carrier of medicines and |
| manufacturing | removal of by-products from |
| | medicines and solvent carriers. |
| Skin treatment products | Hydration of skin cells, improved |
| | absorption of moisturizers, and |
| | reduction in pigment changes due to |
| | sun damage. |
| Wound treatment products | Faster healing and pain relief. |
| Respiratory relief used in | Improved breathing with less snoring. |
| humidifier systems | |
| Eye relief products | Relief for irritated eyes and |
| hydration. | |
| Dental care products | Removal or inhibit plaque and stains |
| | on teeth. |
| Cosmetics and beauty supplies | Less need for chemical binders and |
| | more resistant to contamination |
| | buildup in cosmetics; improved hair |
| | growth. |
| Water features including | Cleaner water with less or no chlorine |
| swimming pools, spas, hot tubs, | and chemical additives. |
| waterfalls, fountains, water | |
| amusement parks | |

Use in Process of Tar Sands Extraction: Conventional water with petroleum solvents used in the separation of tar from sand was replaced with SG Gas-infused Water. SG Gas-infused Water was heated (no petroleum solvent added) with a sample of tar sands in a pan to approximately 160° F. Tar was observed separating from the sand, providing a cleaner and more efficient process with less by-products and emissions released from tar extraction.

Use for Improved Cleaning: For laundry, one may add a quantity (⅓ of a gallon in a standard washing machine tub of 12 gallons for medium load and 16 gallons for large load) of SG Gas-infused Water to the soap cycle of a top loading washing machine and the remaining water (approximately ⅔ of a gallon) is added to the rinse cycle. The polarized characteristic and smaller molecular size of SG Gas-infused Water enable the detergent and water solution to more thoroughly penetrate the cloth fabric and remove the dirt and grime. The addition of SG Gas-infused Water to the rinse assists in completely removing the soap residue that may contain residual dirt from the fabric. This process results in cleaner and stain-free laundry with less body oil and bacteria buildup. Laundry without these SG Gas-infused Water additives display less brilliant whites and retain a pungent odor caused by residual bacteria living in the fabric of the washed clothes.

Reduced Use of Emulsifiers and Surfactants: One may dilute cleaning solutions with SG Gas-infused Water for effective cleaning of surfaces to remove grime, oil and grease and removal of bacteria. SG Gas-infused Water is a natural disinfectant without harsh chemical additives. Typically, one uses at least 1 part cleaning solution with 20 parts SG Gas-infused Water to maintain cleaning properties.

### Biological Properties

Transport, Delivery and Absorption of Nutrients: In a controlled experiment, a standard drug metabolism test in vitro was conducted over a period of 21 days. This comparative test was performed on cell membrane permeability for Vitamin C solution (L-ascorbic acid) using (1) Hank's Buffered Saline Solution (HBSS) and (2) SG Gas-infused Water. Caco-2 cells were used and permeability of the apical side (similar to intestine surface) and basolateral side (similar to underneath intestinal surface) for the separate solutions were determined. Vitamin C quantitiation was conducted on HPLC (HP1100 equipped with PDA detector) and Zorbax C18 reverse phase column (4.6×250 mm, 5 micro) at 30 C. Test results demonstrated Vitamin C permeability of SG Gas-infused Water was about 4 times higher than the control counterpart. (Hu, 2008 (unpublished communication). Results are provided in FIG. 6.

Plant Growth: In a controlled greenhouse setting, four groups of ivy plants were watered using (1) 100% well water, (2) mix of ⅓ mix SG Gas-infused Water and ⅔ well water, (3) mix of ⅔ SG Gas-infused Water and ⅓ well water, and (4) 100% SG Gas-infused Water. The ivy plants were harvested and dehydrated to allow measurement of dry plant mass. The fourth group of 100% SG Gas-infused Water had over 16 percent increase in mass when compared to that the first group of well water. (Reiser, 2006 (private communication).

Fish Growth: Two home aquariums were used to hold two respective groups of goldfish. SG Gas was bubbled into one aquarium and the second with air for a period of thirty days. It was observed that the goldfish in the former aquarium aerated by SG Gas grew at least 15 percent more and the aquarium tank remained cleaner with less algae growth.

Wound Treatment and Healing: The polarization Of the SG Gas-infused Water provides natural anti-bacterial and non-toxic anti-infective properties that promote healing of superficial and multi-layer wounds and a reduction in pain perception. A fifty-year old woman burned herself by accidentally spilling scalding-hot coffee onto her hand. Upon seeking medical attention, a physician advised the patient that she may have to undergo abridement or dead skin removal and possible skin graft surgery. The patient washed the affected area with SG Gas-infused, purified water and applied a medicinal ointment. The wound was wrapped with a sterile gauze and the gauze was moistened to keep the wound hydrated with SG Gas-infused Water. The patient reported an immediate and on-going lessening of pain with the application of SG Gas-infused Water. Over the period of ten days with repeating these treatment steps involving changing of the moistened sterile gauze on at least a daily basis, the site of the wound developed new skin with minimal evidence of scaring.

Upon cessation of the treatment regime when the upper skin layer appeared to be healed, blisters appeared on the surface of the skin. The treatment with SG Gas-infused Water was reinitiated and the blisters healed as well as the remaining layers of skin. The patient experienced healing and thereby avoided debridement of dead skin, and skin grafts. Skin Treatment: Topical applications twice a day on each side of a male volunteer's face in vicinity of his eyes were made. Two types of topical solutions were prepared with 1% magnesium ascorbyl phosphate (MAP), one using SG Gas-infused Water and the other using tap water. After 21 days, the volunteer observed on the side where SG Gas-infused Water solution was applied, a slight reduction in the depth of fine lines around the eye and a lighting of darker skin pigment when compared to that of the other area where the tap water solution was applied. (Puleo of Otima Specialty Chemical, 2008 (private communication).

Eye Relief: SG Gas-infused Water may be sprayed into the eyes for immediate relief and lessening of redness that is comparable to use of over-the-counter eye drops. This natural treatment without any chemical additives, assists in hydrating eyes and removing irritants such as dust and pollen.

Dental Care: A 50:50 solution of commercial mouth wash was mixed with SG Gas-infused Water and a capful of this solution was used twice a day after brushing teeth. Less plaque buildup and stains were noted by professional dental hygienists as compared to previous observations six months earlier when this solution had not been used.

### Molecular Structure Based on Gas Properties

It is believed by the inventors from observing the properties of SG Gas that the process disclosed herein results in a product not achieved by heretofore-reported processes for the electrolysis of water into gas.

Given the low energy reaction that created the gas and the use of no catalysts, it is believed unlikely that any O—H bonds of water could possibly be broken in the process used. It is known that breaking O—H bonds requires two faradays per mole and the process of the invention only employs 2.8 watt hours per liter, which is about a maximum of 1.6 faradays per mole. Further, the SG Gas resulting from the process disclosed herein is flammable but the flame temperature of the gas is only about 270° F. (132.2° C.), as compared to diatomic hydrogen gas which is highly combustible and autoignites at 560° C. A hydrogen/oxygen torch flame is reportedly 3200° C.=5792° F. However, the SG Gas flame easily melts metals, which likely indicates that an oxygen is active. The gas flame also reduces ceramics, which indicates that the hydrogen is in an ionized state.

SG Gas has an affinity for water and other liquids including fuels but bubbles from the liquids after reaching a saturation point. One use of the gas disclosed herein is infusing it back into water to create ionized or polarized water.

SG Gas is always a gas at room temperature while normal water vapor requires energy to evaporate in great quantities. When combusted, the gas always returns to liquid water. When placed in a balloon, the gas initially floats the balloon but it seeps from the balloon rather quickly indicating that the gas has a small molecular structure.

One theory consistent with the properties heretofore observed on SG Gas is that no bonds of H₂O are broken when the process of the invention is used, but that the combination of the electric and magnetic forces restructure the water molecule. Gauss' Law that states there are no monopoles in magnetism, only dipoles. It is well known that liquid water forms hydrogen bonds with other water molecules in order to remain in a liquid solution.

Applying Gauss' Law to hydrogen, it has polar properties that opens up a new configuration, one in which a hydrogen can be bound to another hydrogen and an oxygen. Upon exposure to an electric current, the electronegative strength of the oxygen atom is weakened, allowing a hydrogen atom to dislodge and magnetically bond to the other hydrogen atom that is strengthened by the magnetic field. Hence, the electric and magnetic forces made possible a shift of a hydrogen from H—O—H to O—H—H creating a diatomic hydrogen molecule that is single bonded to atomic oxygen. As the exposed oxygen is a reactive site on the gas molecule an appropriate name is “hydroxyhydrogen”. This structure predicts that the oxygen is now active and can oxidize metals. It predicts that in the unburned gaseous state, the increased negative charge causes greater spacing among the gas molecules causing stability, a lower boiling point, a lower freezing point, and a higher vapor pressure.

The inventors have conceived of a new isomer of water—it contains the same atoms, only in a different configuration and thus exhibits different properties from normal water vapor. The gas does not cluster to create liquid water at regular atmospheric temperatures and pressures as does the molecules of normal water vapor. The gas exists in a higher energy state, burns by itself at a low temperature, and melts any substrates when exposed to the gas flame. The gas flame has a uniform blue color appearance without yellow sparks indicative of water (H₂O) vapor or red sparks indicative of either H₂ or O₂ gas contamination. Hence, we call the resulting gas (SG Gas) an ionized gas or a plasma gas.

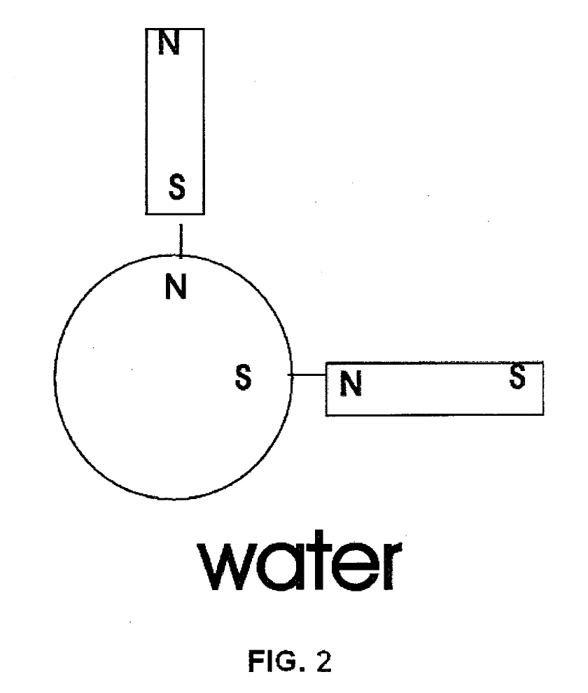

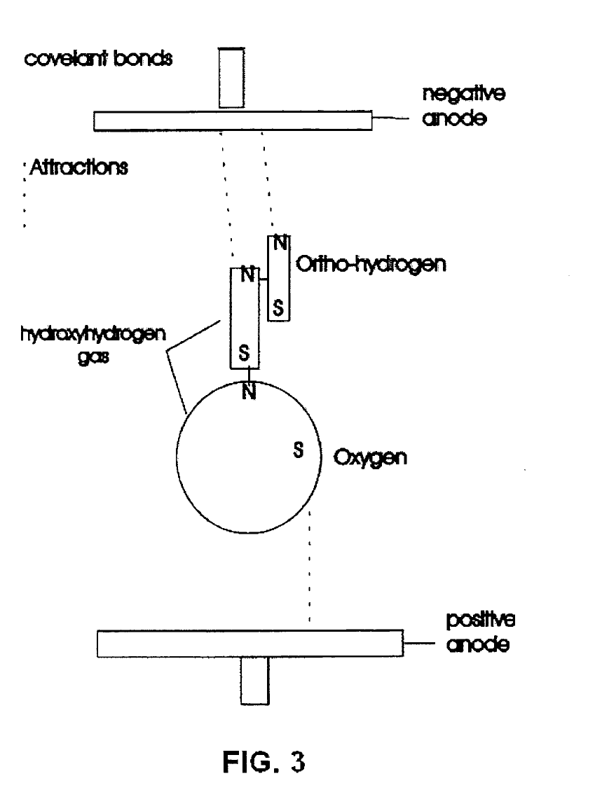

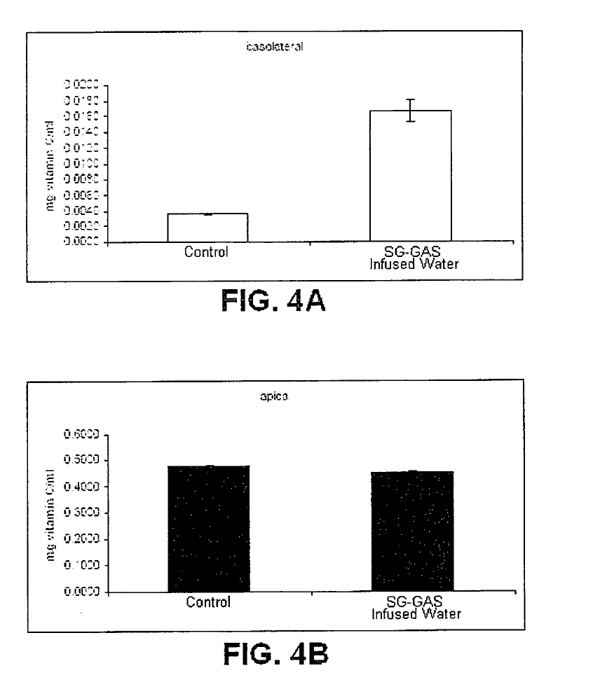

Now referring to FIGS. 2-3, atoms shown are shown in their polar orientation for better understanding N meaning North Pole and S meaning South Pole. This dictates the orbital spin or magnetic flux. FIG. 2 illustrates water prior to undergoing the process of the invention. FIG. 3 illustrates the process and the believed effect on the aqueous fluid used.

While the magnetic field orients the atoms within the water molecule, the collapsing field induces a charge in the opposite direction that dislodges the opposing hydrogen bond and allows it to bond to the other hydrogen atom in the ortho position as depicted in FIG. 3. Ortho-hydrogen is more reactive than para-hydrogen and produces much more energy.

This reaction changes water from a liquid cluster to an ionized gas or plasma gas that will, when ignited, and the flame applied to a solid substrate, melt nearly any substance. Further, when the gas is infused into a water cluster it will bond to the water molecules and create a much smaller cluster of a different shape and properties allowing it to penetrate cells and hydrate animals and plants at a substantially faster rate.

It must be clear that due to the process used herein, electrolysis does not take place. “Electrolysis” is defined as a “method of separating chemically bonded elements and compounds by passing an electric current through them.” Electrolysis does not take place and no splitting of the water molecular bonds occurs, as is demonstrated by the fact that no increase in hydrogen or oxygen gas can be measured in the reaction zone. This is a key differentiator from the processes that have resulted in a gas being produced by electrolysis of water. The gases produced by electrolysis exhibit far different properties from SG Gas. Gases produced by electrolysis are explosive, cannot be pressurized and are heat-producing gases on ignition.

SG Gas is herein disclosed to be an ionized gas with the potential to oxidize or reduce any substance. On a non-oxidized substrate, such as steel, the active oxygen within the molecule will chemically bond to the steel bringing it immediately to its melting temperature and releasing hydrogen, which bonds with atmospheric oxygen to produce heat. On an oxidized substrate, such as ceramic, the hydrogen reduces the substrate by chemically bonding with the oxygen present within the substrate, melting the material and releasing atomic oxygen, which then bonds with the material. This double reaction is responsible for producing much more heat than an ordinary oxidation reduction reaction.

These reactions are proven on rusty steel and concrete. When ordinary gas, such as: methane, ethane, propane, butane, or acetylene are applied to rusty steel popping and spitting of material occurs due to the explosive reaction of the ferrous oxide being separated from the non-oxidized metal due to different expansion rates. With SG Gas, this does not occur, since oxidation and reduction are occurring at the same time and the expansion rates are equal. On concrete when heat from an ordinary gas is applied, the portion the flame touches will expand and break loose from the rest of the concrete with an explosive force and spit pieces of hot concrete outward and leave holes in the concrete surface. Again, this does not occur with SG Gas because it is being reduced to a liquid form before the pressure of uneven expansion occurs.

Simply stated SG Gas is an ionized gas capable of oxidizing or reducing almost any material without the adverse reactions created by heat producing flames. Heat is the byproduct of friction, in chemistry two atoms colliding together in a reaction known as oxidation and reduction cause this friction. A gas, referred to as a fuel, is usually a hydrocarbon that is easily oxidized, however, the carbon is what is being oxidized and the oxygen is being reduced meaning this is where friction occurs and these are the items being heated. Heat given off by these substances is refractive heat and the substances being heated are absorbing heat or, better stated, are being bombarded by fast moving hot gases. SG Gas may change the definition of melting point due to the lack of heat producing flames.

1. A method for making a gas comprising the steps of:
(a) providing a volume of aqueous fluid to a reaction zone comprising a receptacle and a gas output means, wherein said reaction zone is closed and may withstand pressure of at least 30 psi;
(b) providing a magnetic field to said reaction zone under conditions which will not induce electrolysis of said aqueous fluid;
(c) collapsing said magnetic field periodically, whereby a generated gas is formed in said reaction zone; and
(d) collecting said generated gas.

2. The method of claim 1, wherein said magnetic field is provided by means of two opposing metallic plates spaced at least one inch apart to which a direct current is applied which pulses and periodically collapses said magnetic field, whereby a generated gas is formed in an area of said aqueous fluid between said opposing electrodes.

3. The method of claim 2, wherein said opposing electrodes are spaced between one inch apart and twenty feet apart.

4. The method of claim 3, wherein said opposing electrodes are spaced from about one inch to about sixteen inches apart.

5. The method of claim 1, wherein said magnetic field is provided by means of two wires in contact with said aqueous fluid, said wires in conjunction with said aqueous fluid comprising an electrical circuit when current is applied thereto.

6. The method of claim 1, wherein a primary and a secondary magnetic field are provided to said aqueous fluid, said primary magnetic field provided by a wire coil outside the reaction zone to which a source of DC power is supplied to create said primary magnetic field, and said secondary magnetic field provided by means of a wire coil placed in the middle of the aqueous fluid solution, which when powered in the opposite direction of the current flow in pulses, collapses the primary field thereby creating the necessary conditions to form the gas.

7. The method of claim 1, further comprising the step of compressing the generated gas and storing in a pressurized container.

8. The method of claim 7, wherein said generated gas is compressed to less than 1,600 psi.

9. The method of claim 8, wherein said generated gas is pressurized to about 1,000 psi.

10. The method of claim 9, wherein said generated gas is storable for at least 30 days.

11. The method of claim 1, wherein said aqueous fluid is an electrolytic fluid comprising a salt dissolved in distilled water, said salt selected from the group consisting of potassium hydroxide, lithium hydroxide and sodium hydroxide and having a specific gravity greater than 1.0 and up to 1.2.

12. The method of claim 1, wherein the conditions in the reaction zone are such that the temperature remains less than about 30° F. above ambient temperature.

13. The method of claim 1, wherein the conditions in the reaction zone are such that the pressure does not exceed 100 psi.

14. The method of claim 1, further comprising exposing the generated gas to a second magnetic field by providing a second reaction zone comprising rare earth magnets.

15. The method of claim 14, wherein said rare earth magnets have a strength greater than fifty (50) Gauss units.

16. The method of claim 15, wherein said generated gas flows through said second reaction zone comprising said rare earth magnets.

17. The method of claim 14, wherein said rare earth magnets comprise a composite of neodymium, iron and boron.

18. The method of claim 17, wherein said rare earth magnets further comprise a nickel coating or plating.

19. The generated gas product of the process of any of the above claims.

20. A pressurized generated gas product which is at least 1000 psi and which is odorless, colorless under visible light, blue gray under an ultraviolet light, and is flammable when exposed to an igniting source.

21. The generated gas product characterized by producing a very low temperature flame of approximately two hundred seventy degrees Fahrenheit (270° F.) upon being ignited.

22. A method of melting materials by igniting SG Gas and putting the resulting flame in contact with said materials.

23. The method of claim 22 wherein said gas is characterized when burning in contact with a substance, causing a chemical reaction such that the substance rises to its intrinsic melting or oxidation point.

24. The method of claim 23, wherein said substance is selected from stainless steel having an intrinsic oxidation point of 2,600° F., steel having an intrinsic oxidation point of 1,330° F., and copper having an intrinsic oxidation point of 1,984° F. and which melt when exposed to the SG Gas flame for less than one minute.

25. The method of claim 23, wherein said substance is selected from ceramics and silicon dioxide.

26. A method for generating heat useful for transfer to a fluid selected from air or water, thereby producing hot air or steam for production of mechanical energy, comprising contacting a substrate with a SG Gas flame under conditions that permit transfer of heat from a reaction of said SG Gas with said substrate to said fluid.

27. The method of claim 26, wherein said substrate comprises a conduit through which said fluid is flowing at the time of contact with said SG Gas flame, and whereby said fluid is heated and flows away from the reaction site to a site where it is used to generate mechanical energy or heat.

28. A method for infusing SG Gas into an air intake of combustion engine generating more efficient burn of fuel, thereby producing extending the energy of said fuel used for mechanical energy, comprising contacting SG Gas with said fuel under conditions that improve chemical reaction of fuel during its combustion cycle thru generating more energy from said fuel.

29. The method of claim 28, wherein said combustion engine comprises a conduit through which said fuel is flowing at the time of contact with said SG Gas, and whereby said fuel combusts in reaction site where said fuel more efficiently combusts to generate mechanical energy and less harmful emissions.

30. Water infused with SG Gas.

31. A method for using water infused with SG Gas for ingestion.

32. A method for using water infused with SG Gas for providing improved growth to biological systems.

33. A method for using water infused with SG Gas for improving uptake of nutrients by animal cells.

34. The method of claim 33, wherein said nutrients is a vitamin.

35. The method of claim 34, wherein said vitamin is Vitamin C.

36. A method for using SG Gas-infused Water for improving uptake of fertilizers by plant cells.

37. A method for topical application of SG Gas-infused water to skin.

38. The method of claim 37, wherein said skin is broken and in need of healing, and application of SG Gas-infused Water accelerates healing of said skin.

39. The method of claim 37, wherein said skin is dehydrated, and application of SG Gas-infused Water provides hydration of said skin.

40. A method for treatment of the eye for irritation or lack of hydration, comprising applying SG Gas-infused Water to said eye for removal of irritants and improved visual clarity.

41. A method for reducing the amount of surfactant or emulsifiers in a cleaning formulation, comprising adding SG Gas-infused Water to aid cleaning formulation.

42. A method for cleaning, comprising application of SG Gas-infused Water to a surface as a cleaning agent.

43. The method of claim 42, wherein said surface comprises teeth.

44. The method of claim 43, further comprising adding a mouthwash to said SG Gas infused water for removal of plaque and stains on teeth.

45. (canceled)
